(19) United States
(12) Patent Application Publication
(10) Pub. No.: US 2021/0251975 A1
(43) Pub. Date: Aug. 19, 2021

(54) USE OF CICLOPIROX FOR INHIBITING HBV CORE ASSEMBLY

(71) Applicants: GWANGJU INSTITUTE OF SCIENCE AND TECHNOLOGY, Gwangju (KR); CHA INIVERSITY INDUSTRY-ACAMEMIC COOPERATION FOUNDATION, Pocheron-si, Gyeonggi-do (KR); SEOUL NATIONAL UNIVERSITY HOSPITAL, Seoul (KR)

(72) Inventors: Sung Gyoo PARK, Gwangju (KR); Jung Ah KANG, Gwangju (KR); Yuri CHO, Seoul (KR); Yoon Jun KIM, Seoul (KR)

(21) Appl. No.: 17/270,770

(22) PCT Filed: Aug. 23, 2018
(86) PCT No.: PCT/KR2018/009741

§ 371 Date: Feb. 23, 2021

(51) Int. Cl. A61K 31/4418; A61K 31/675; A61K 31/522; A61P 31/20

(52) U.S. Cl. CPC A61K 31/4418; A61P 31/20; A61K 31/522; A61K 31/675

(57) ABSTRACT

The present invention relates to an anti-hepatitis B virus (HBV) composition containing ciclopirox or a pharmaceutically acceptable salt thereof; a pharmaceutical composition for preventing or treating an HBV-induced disease, which contains ciclopirox or a pharmaceutically acceptable salt thereof; a method for treating an HBV-induced disease, which includes a step of administering the pharmaceutical composition to a subject; and a health functional food composition for preventing or improving an HBV-induced disease, which contains ciclopirox or a physiologically acceptable salt thereof.

The present invention has newly elucidated the HBV-inhibiting effect of ciclopirox, and overcame the problem of the existing drugs that cccDNA cannot be removed by monotherapy. In addition, the present invention provides a therapeutic agent capable of effectively inhibiting HBV by inhibiting core assembly during the life cycle of the virus. Furthermore, the occurrence of diseases such as chronic hepatitis B, hepatocirrhosis, and hepatocellular carcinoma can be decreased.

### TECHNICAL FIELD

The present invention elucidates a novel drug effect of ciclopirox and specifically relates to an anti-hepatitis B virus (HBV) composition containing ciclopirox or a pharmaceutically acceptable salt thereof; a pharmaceutical composition for preventing or treating an HBV-induced disease, which contains ciclopirox or a pharmaceutically acceptable salt thereof; a method for treating an HBV-induced disease, which includes a step of administering the pharmaceutical composition to a subject; a health functional food composition for preventing or improving an HBV-induced disease, which contains ciclopirox or a physiologically acceptable salt thereof; etc.

### BACKGROUND ART

Hepatitis B virus (HBV) infection is a very important health issue because it has a high incidence rate globally and about 6% to 10% of HBV infection is highly likely to develop into chronic liver diseases such as hepatocirrhosis or hepatocellular carcinoma. In Korea, the incidence rate of hepatocellular carcinoma is 22.2 people (36.0 males and 10.2 females) out of 100,000 people, and the mortality from hepatocellular carcinoma is 15.4 people (25.8 males and 6.6 females) out of 100,000 people (Korea Centers for Disease Control and Prevention).

In the early interferon (IFN) therapy for inhibiting HBV which induces such diseases, HBV infection was treated by activating cytotoxic T lymphocytes and thereby enhancing immune responses. High response rates were observed when the duration of disease was short, the serum aminotransferase level was high, or when the hepatitis B virus DNA level was low. In addition, there are advantages in that the replication of HBV can be inhibited through inhibited transcription of covalently closed circular DNA (cccDNA) and that the activation of NK cells can be regulated. However, there are problems of severe side effects such as fever, chill, general weakness, depression, congestive heart failure, neutropenia, etc.

It has been found that lamivudine (3-TC), which was developed as a therapeutic agent for AIDS that can avoid the problems of interferon, is effective for treatment of HBV. Although lamivudine is an effective medication for treatment of chronic hepatitis B, there is a problem of the emergence of lamivudine-resistant HBV after long-term use.

Recently, entecavir (ETV) or tenofovir (TDF), which shows highly potent antiviral effect for treatment of hepatitis B and shows few resistance problems, are used. These oral antiviral medications which inhibit the reverse transcription of virus showed improved therapeutic performance with few side effects. However, these medications, which inhibit the replication of virus as nucleotide analogues, cannot completely remove the virus and are not applicable to long-term treatment because the cccDNA in the nucleus cannot be removed. Therefore, a new therapeutic agent capable of completely removing HBV is necessary.

Meanwhile, HBV is a double-stranded DNA virus. After infection, RNA polymerase produces polymerase, a coat protein, and an HBx protein from a DNA template. From a single infected cell, 200 to 300 new hepatitis B viruses are produced by genome. Therefore, a large quantity of viruses are produced and released. HBx is known as a representative pathogenic protein. Although it does not bind directly to DNA, it is known to act as a transactivator and affect interaction with immune response-related proteins and various signal transductions.

Ciclopirox is known as a hydroxypyridinone antifungal agent and is studied as a therapeutic agent for seborrhoeic dermatitis. However, nothing is known about its therapeutic effect for HBV.

Under this background, the inventors of the present invention have made extensive efforts to solve the above-described problems. As a result, they have newly identified the HBV-inhibiting effect of ciclopirox and have completed the present invention.

### DISCLOSURE

### Technical Problem

An object of the present invention is to provide an anti-hepatitis B virus (HBV) composition, which contains ciclopirox or a pharmaceutically acceptable salt thereof.

Another object of the present invention is to provide a pharmaceutical composition for preventing or treating an HBV-induced disease, which contains ciclopirox or a pharmaceutically acceptable salt thereof.

Still another object of the present invention is to provide a method for treating an HBV-induced disease, which includes a step of administering the pharmaceutical composition to a non-human subject.

Still another object of the present invention is to provide a health functional food composition for preventing or improving an HBV-induced disease, which contains ciclopirox or a physiologically acceptable salt thereof.

### Technical Solution

Hereinafter, the present invention is described more specifically. Each description and embodiment in the present invention may also be applied to other descriptions and embodiments. That is to say, all the combinations of various elements disclosed herein fall within the scope of the present invention. Further, the scope of the present invention is not limited by the specific description given below.

In an aspect, the present invention provides an anti-hepatitis B virus (HBV) composition containing ciclopirox or a pharmaceutically acceptable salt thereof.

The ciclopirox is a compound represented by following Chemical Formula 1. Although it is known as an antifungal agent and is studied as a therapeutic agent for seborrheic dermatitis, nothing is known about its therapeutic effect for HBV.

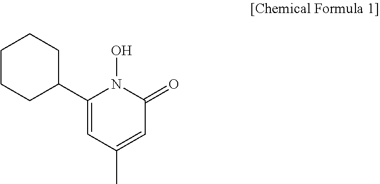

The inventors of the present invention have newly identified that ciclopirox specifically inhibits the core assembly process during the life cycle of HBV. Specifically, the inventors of the present invention have made efforts to solve the disadvantage of the existing drugs such as entecavir, tenofovir, etc. that they cannot remove the cccDNA of HBV, and, as a result, have newly identified that ciclopirox can effectively remove the cccDNA and inhibit the core assembly of HBV.

Specifically, it was identified that ciclopirox inhibits the assembly of a purified HBV core protein in assembly environment, and that ciclopirox inhibits the assembly also in the cells overexpressing the core or full-length DNA of HBV. In addition, it was identified that, when the purified core protein was isolated depending on morphology according to a sucrose concentration gradient, assembled cores were decreased and cores in dimer forms were increased due to ciclopirox (FIG. 2).

More specifically, as a result of structural analysis of the HBV core protein binding to ciclopirox, it was identified that ciclopirox binds to the core protein and, especially that tyrosine 118 is important for the binding (FIG. 3). In addition, it was identified from this that ciclopirox inhibits core assembly by binding directly to the HBV core protein.

The ciclopirox of the present invention reduces HBV in HBV-expressing cell lines and cell lines in which HBV is expressed arbitrarily according to a concentration gradient of the drug, without affecting cell viability (FIG. 5). In addition, it was confirmed that not only the amount of DNA released out but also the amount of DNA remaining inside is decreased when HBV-expressing cell lines are treated with ciclopirox according to the concentration gradient of the drug (FIG. 6).

The ciclopirox may inhibit the assembly of the HBV core protein. More specifically, it may inhibit the core assembly by binding to a tyrosine residue (tyrosine 118) or a tryptophan residue (tryptophan 102), which is essential in the core assembly step.

In the present invention, “anti-HBV” refers to the action of specifically inhibiting the proliferation of HBV virus by specifically inhibiting the cytopathic effect by the HBV virus.

In the present invention, hepatitis B virus (HBV) refers to a DNA virus causing hepatitis B and is also called HBs. The hepatitis B virus contains DNA, DNA polymerase, an HBc antigen, and an HBe antigen in the central core.

The anti-HBV composition may further contain entecavir, tenofovir, or a combination thereof.

It was confirmed that the ciclopirox of the present invention exhibits synergistic effect when treated together with entecavir or tenofovir as compared to when treated alone, and that reduces not only the DNA released out but also the DNA remaining inside is decreased according to the concentration gradient of the drug (FIG. 7).

In another aspect, the present invention provides a pharmaceutical composition for preventing or treating an HBV-induced disease, which contains ciclopirox or a pharmaceutically acceptable salt thereof.

In still another aspect, the present invention provides a method for treating an HBV-induced disease, which includes a step of administering the pharmaceutical composition to a subject.

The ciclopirox, the pharmaceutically acceptable salt, and the HBV virus are the same as described above.

In the present invention, the HBV-induced disease refers to a disease that may be caused by HBV infection. Examples thereof may include hepatitis, hepatocirrhosis, hepatocellular carcinoma, or a combination thereof, but are not limited thereto.

The pharmaceutical composition may further contain entecavir, tenofovir, or a combination thereof.

As used herein, the term “prevention” refers to any action of inhibiting or delaying an HBV infection disease by administering the composition of the present invention. In addition, as used herein, the term “treatment” refers to any action of improving or favorably changing the symptoms of an HBV-induced disease by administering the composition.

As used herein, the term “administration” refers to introducing the pharmaceutical composition of the present invention to a subject by any suitable means. In addition, the composition of the present invention may be administered via various oral or parenteral administration routes that can reach the target tissue.

As used herein, the term “subject” refers to any animal including human in which an HBV infection disease has already occurred or can occur. The disease can be prevented and treated effectively by administering the composition of the present invention to the subject.

The composition of the present invention is administered with a pharmaceutically effective amount. As used herein, the term “pharmaceutically effective amount” refers to an amount sufficient to treat a disease at a reasonable benefit/risk ratio applicable to medical treatment. An effective dosage level may be determined depending on the type of the subject, the severity of a disease, the age and sex of the subject, the type of the virus infection disease, drug activity, sensitivity to the drug, administration time, administration route, excretion rate, the duration of treatment, drugs used in combination, and other factors well known in the medical field. The composition of the present invention may be administered as an individual therapeutic agent or may be administered in combination with another therapeutic agent. The co-administration with the existing therapeutic agent may be made sequentially or simultaneously. In addition, the administration may be made once or multiple times. It is important to administer the composition with the minimum amount that can achieve the maximum effect without causing side effects, in consideration of all the above-described factors, and the amount can be easily determined by those skilled in the art.

The pharmaceutical composition of the present invention may further contain, in addition to the above-described active ingredient, a pharmaceutically acceptable carrier, an excipient, or a diluent. Examples of the carrier, excipient, or diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

The pharmaceutical composition of the present invention may be formulated into an oral formulation (e.g., a powder, a granule, a tablet, a capsule, a suspension, an emulsion, a syrup, an aerosol, etc.) a formulation for external application, a suppository or a sterilized injection solution according to common methods. Specifically, the formulations may be prepared using a commonly used diluent or excipient such as a filler, an extender, a binder, a wetting agent, a disintegrant, a surfactant, etc. Solid formulations for oral administration include a tablet, a pill, a powder, a granule, a capsule, etc., but are not limited thereto. These solid formulations may be prepared by mixing with at least one excipient, e.g., starch, calcium carbonate, sucrose, lactose, gelatin, etc. Furthermore, lubricants such as magnesium stearate and talc may be used in addition to the simple excipients. Liquid formulations for oral administration may be prepared by adding various excipients, e.g., a wetting agent, a sweetener, an aromatic, a preservative, etc. in addition to liquid paraffin. Formulations for parenteral administration include a sterilized aqueous solution, a nonaqueous solution, a suspension, an emulsion, a lyophilized formulation, and a suppository. In the nonaqueous solution or suspension, propylene glycol, polyethylene glycol, a vegetable oil such as olive oil, an injectable ester such as ethyl oleate, etc. may be used. As a base of the suppository, witepsol, macrogol, Tween 61, cocoa butter, laurin butter, glycerogelatin, etc. may be used.

The pharmaceutical composition of the present invention may be administered orally or parenterally (e.g., intravenously, subcutaneously, intraperitoneally, or topically) depending on the intended use. The administration dosage may vary depending on the patient's condition and body weight, the severity of a disease, the type of the drug, and the route and time of administration. In general, a daily dosage is about 50 mg/kg, preferably 20 mg/kg to 100 mg/kg. The administration may be made several times, preferably 1 to 4 times, a day depending on the discretion of a physician or a pharmacist, and may be adequately determined by those skilled in the art.

In still another aspect, the present invention provides a health functional food composition for preventing or improving an HBV-induced disease, which contains ciclopirox or a physiologically acceptable salt thereof.

The ciclopirox, the salt, and the HBV virus are the same as described above.

The ciclopirox or a physiologically acceptable salt thereof may be added to the health functional food composition for the purpose of preventing or improving HBV infection. When the ingredient is used as a health functional food additive, it may be added either alone or in combination with another food or food ingredient according to common methods. The mixing amount of the active ingredient may be determined adequately depending on purposes (prevention, health improvement, or therapeutic treatment).

As used herein, the term “improvement” may refer to any action that at least reduces a parameter related to the condition to be treated, for example, the degree of symptoms.

As used herein, the term “health functional food” refers to a food prepared or processed into the form of a tablet, a capsule, a powder, a granule, a liquid, a pill, etc. using a material or an ingredient having a functionality useful for the human body. In particular, the functionality refers to an effect useful for regulation of nutrients for the structure or function of the human body or health care such as physiological actions, etc. The health functional food of the present invention may be prepared by methods commonly used in the art, and may contain materials and ingredients commonly used in the art. In addition, it may have the advantage that there is no side effect, etc. that may occur in long-term medication of a drug, because it is prepared from food materials unlike general drugs, and may have excellent portability.

When the composition of the present invention is used and contained in a health functional food, the composition may be added either alone or in combination with another health functional food or health functional food ingredient, and may be used according to common methods. The mixing amount of the active ingredient may be determined adequately depending on the purpose of use (prevention, health improvement, or therapeutic treatment). In general, the composition of the present invention is added in an amount of 1 wt % to 10 wt %, specifically 5 wt % to 10 wt %, relative to the raw materials of food. However, for long-term intake not intended for health or hygiene improvement, the addition amount may be decreased to the above-described range.

The health functional food composition may further contain entecavir, tenofovir, or a combination thereof.

### Advantageous Effects

The present invention has newly elucidated the HBV-inhibiting effect of ciclopirox, which is a drug with proven safety, and overcame the problem of the existing drugs that cccDNA cannot be removed by monotherapy. In addition, the present invention provides a therapeutic agent capable of effectively removing HBV by inhibiting core assembly during the life cycle of the virus. Furthermore, the occurrence of diseases such as chronic hepatitis B, hepatocirrhosis, and hepatocellular carcinoma can be decreased.

### BRIEF DESCRIPTION OF DRAWINGS

FIG. 1 shows a procedure of investigating the HBV-inhibiting effect of ciclopirox. A in FIG. 1 schematically illustrates the screening of drugs having the HBV-inhibiting effect from about 1,000 drugs; B in FIG. 1 shows the HBV-inhibiting effect of 19 drugs selected through the first screening; C in FIG. 1 shows the HBV transcript expression level of the 19 drugs; and D in FIG. 1 shows the ability of the 19 drugs for inhibiting the expression of core and capsid proteins. Results were expressed as mean±standard deviation and tested by the Student's t-test. *: p<0.05, ** p<0.01.

FIG. 2 shows the core assembly-inhibiting ability of ciclopirox. A in FIG. 2 shows immunoblotting images exhibiting the core assembly-inhibiting ability of ciclopirox; and C and D in FIG. 2 show the core assembly-inhibiting ability of ciclopirox when the core protein was expressed in liver cells and when the cells were treated with ciclopirox. Specifically, there was no change in the amount of the reduced core protein on SDS-PAGE gel, but the assembled core protein was significantly decreased on native gel. B in FIG. 2 shows a result of investigating the purified core protein in the same manner as in A in FIG. 2 using a sucrose concentration gradient. It can be seen that core assembly was decreased.

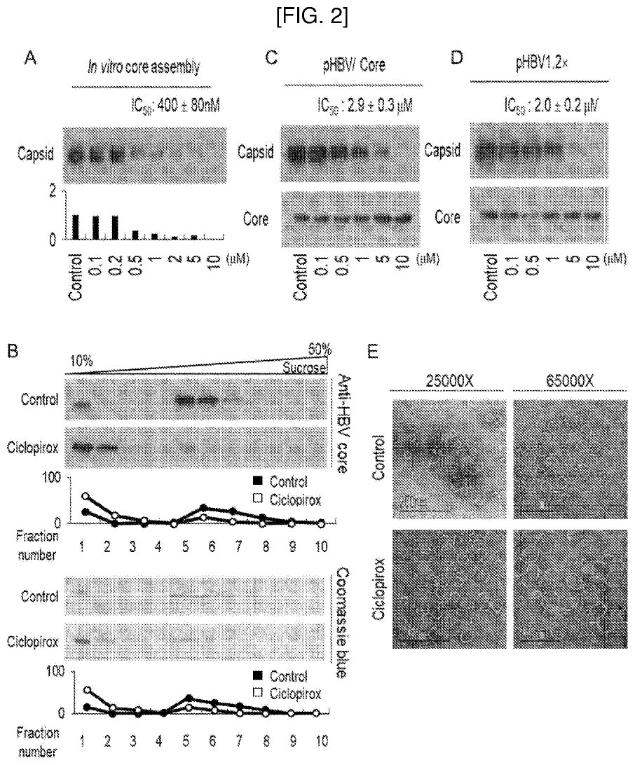

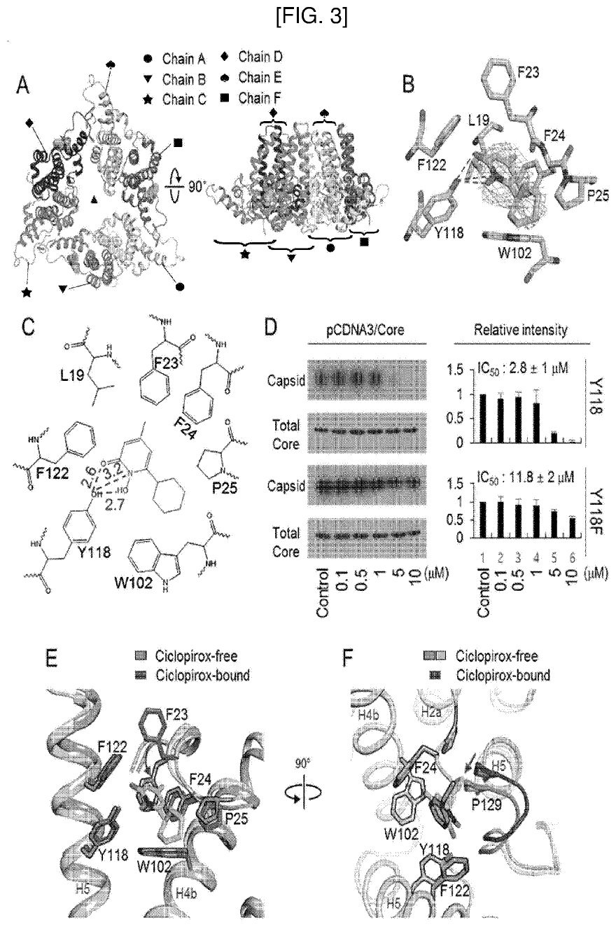

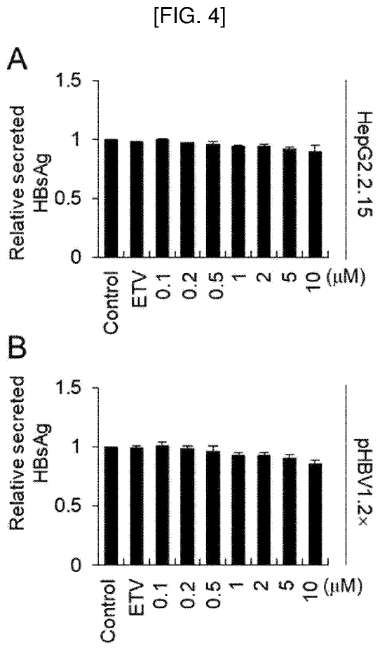

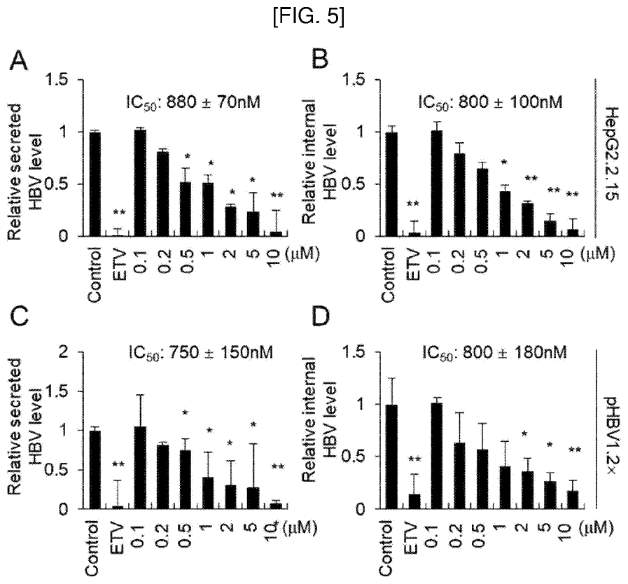

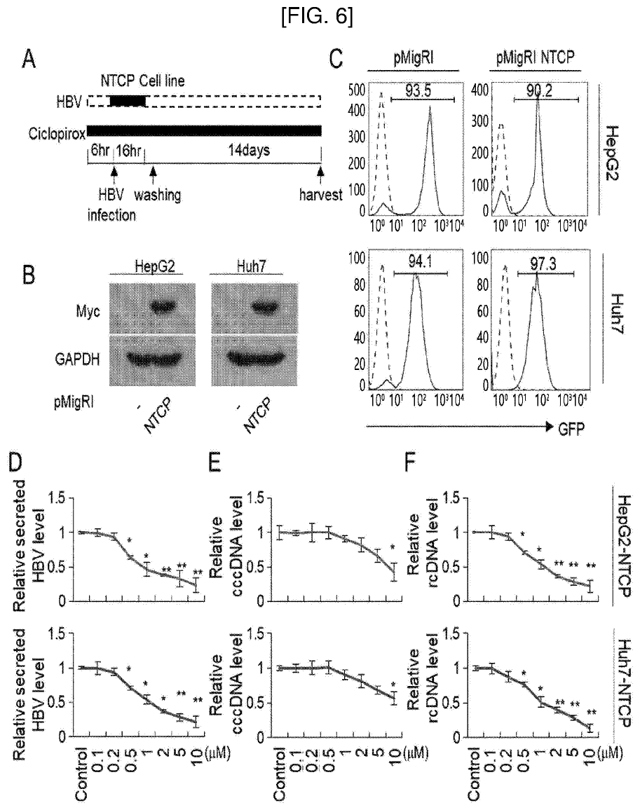

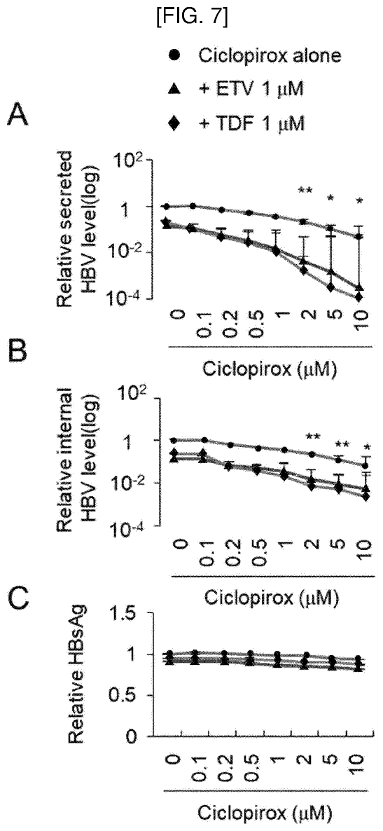

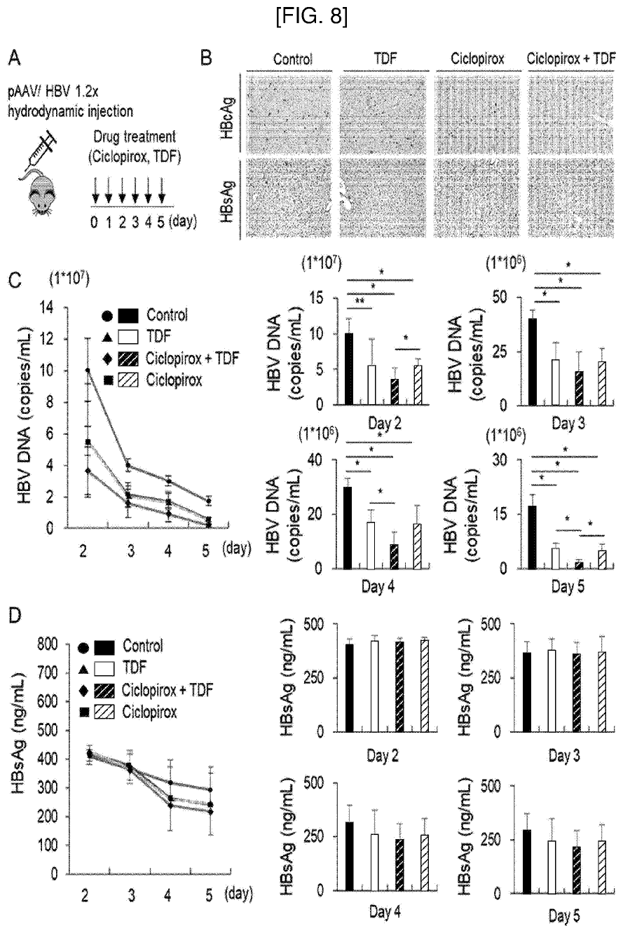

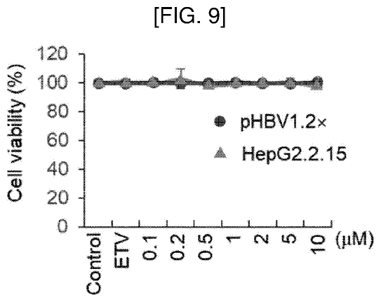

Specifically, B in FIG. 2 shows a result of preparing a sucrose concentration gradient from 10% to 50% based on the change in a core protein after core assembly depending on the sucrose concentration gradient and separating the assembled and drug-treated core protein by ultracentrifugation. It can be seen that the assembled core protein was decreased and the unassembled protein in dimeric form was remarkably increased. C and D in FIG. 2 show the core assembly-inhibiting ability of ciclopirox when the core protein of HBV was expressed and when the entire protein of HBV was expressed, respectively. Although there was no change in the amount of the individual core protein, the assembled core was decreased significantly by ciclopirox. E in FIG. 2 shows electron microscopic images showing that the size of the assembled core is enlarged by ciclopirox and the circular shape is destroyed. In summary, FIG. 2 shows that ciclopirox exhibits HBV-inhibiting effect by inhibiting the assembly of the HBV core protein.

FIG. 3 shows a result of analyzing the site where ciclopirox binds to the HBV core protein. Specifically, A in FIG. 3 shows the overall hexagonal structure of an asymmetric unit. The secondary structure of the protein was computed using STRIDE. The space-filling model shows the binding of ciclopirox to the core protein. B and C in FIG. 3 shows the sites where ciclopirox binds to the HBV core protein. In particular, the hydrogen bonding at tyrosine (Y) 118 is important. D in FIG. 3 shows that mutation at Y118 reduces inhibition of core assembly by ciclopirox. E and F in FIG. 3 show the ciclopirox-bound and ciclopirox-free sites of chains B and C.

FIG. 4 shows a result of quantifying the amount of the HbsAg protein secreted from HBV-expressing cell lines and HBV-overexpressing cell lines. Specifically, A and B in FIG. 4 show a result of an enzyme-linked immunosorbent assay, which shows the change in the amount of released HBsAg after treatment with ciclopirox. As a result, it was found that there was no significant change in HBsAg depending on the ciclopirox concentration gradient. Results were expressed as mean±standard deviation and tested by the Student's t-test. *: p<0.05, ** p<0.01.

FIG. 5 shows the HBV-inhibiting effect of ciclopirox depending on a concentration gradient, at 7 concentrations of 0.1 mM, 0.2 mM, 0.5 mM, 1 mM, 2 mM, 5 mM, and 10 mM. Specifically, A and B in FIG. 5 show a result of treating HepG2.2.15 cells and HBV-expressing liver cells with ciclopirox for 6 days and investigating the decrease of HBV DNA at different concentration gradients. C and D FIG. 5 show a result of investigating the inhibitory effect of ciclopirox by extracting HBV DNA from the cells rather than the DNA secreted out of the cells. Specifically, after lysing HepG2.2.15 cells and HBV-expressing liver cells and removing capsidated RNA using micrococcal nuclease, virus DNA was extracted and its expression level was investigated. As a result, it was confirmed that HBV DNA was decreased according to the concentration gradient of ciclopirox.

FIG. 6 shows a result of cells infected with HBV using an HBV infection system and investigating the HBV-inhibiting effect of ciclopirox. A in FIG. 6 shows the flow of the HBV infection system. Specifically, after treating HepG2 and Huh7 cell lines which express a receptor (NTCP) essential for HBV infection with ciclopirox for 6 hours, the supernatant of HepG2.2.15 cells were treated with ciclopirox. 16 hours later, after viral washing, followed by treating with ciclopirox every day for 14 days, the cells and cell supernatant were collected and analyzed. B and C in FIG. 6 show a result of investigating the expression of NTCP in the NTCP cell lines by immunoblot and flow cytometry. D to F in FIG. 6 show the HBV-inhibiting effect of ciclopirox in the virus infection system. Specifically, D in FIG. 6 shows that the HBV DNA secreted out of the two cell lines is decreased significantly depending on the concentration of ciclopirox. E in FIG. 6 shows that, as a result of investigating the expression level of cccDNA in which rcDNA is removed, the expression level was decreased significantly in the two cell lines depending on the concentration of ciclopirox. F in FIG. 6 shows that, as a result of investigating the rcDNA expression level in the ells, the expression level thereof was decreased significantly in the two cell lines depending on the concentration of ciclopirox.

FIG. 7 shows the potentiality of combination treatment of ciclopirox. It can be seen that ciclopirox treated in combination with entecavir (ETV) and tenofovir (TDF) shows synergistic HBV-inhibiting effect. After treating with ciclopirox according to a concentration gradient, 1 mM ETV or 1 mM TDF was treated in combination. Specifically, A in FIG. 7 shows a result of treating with the drugs for 6 days and then quantifying the secreted HBV DNA. As a result, HBV DNA was reduced greatly when ciclopirox was treated together with ETV or TDF as compared to when it was treated alone. B in FIG. 7 shows a result of quantifying the amount of HBV DNA existing in the cells under the same concentration. It was confirmed that HBV DNA was reduced greatly when ciclopirox was treated together with ETV or TDF as compared to when it was treated alone. C in FIG. 7 shows a result of measuring the quantity of the HBsAg protein secreted out of the cells. No HBsAg-inhibiting effect was observed when treated with ciclopirox alone or in combination with ETV or TDF.

FIG. 8 shows HBV-inhibiting effect when ciclopirox was injected into HBV-expressing mouse. A in FIG. 8 shows a procedure of expressing HBV in mouse and treating with ciclopirox, TDF, or a combination of ciclopirox and TDF every day for 5 days. Specifically, after expressing HBV in mouse by injecting using a hydrodynamic injection method an HBV-expressing pAAV HBV 1.2× plasmid into the mouse tail, the drug was treated every day for 5 days. B in FIG. 8 shows that, when the expression level of an HBV core protein and surface protein in liver cells was investigated after the drug treatment, ciclopirox reduced the quantity of the HBV core protein and the treatment in combination with TDF decreased the quantity more effectively. C in FIG. 8 shows that, when the amount of HBV DNA contained in blood was quantitated after treating with the drug, ciclopirox reduced the quantity of HBV DNA and the treatment in combination with TDF decreased the quantity more effectively. D in FIG. 8 shows a result of quantifying the amount of the HBsAg protein contained in blood after treating with the drug.

FIG. 9 shows an MTS result for investigating cell viability for ciclopirox. As a result, it was confirmed that ciclopirox had no effect on the cell viability of HBV-expressing HepG2.2.15 cell lines and HBV-expressing liver cell lines.

### DETAILED DESCRIPTION OF THE INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### Experimental Example 1. Cell Culturing

HegG2, HepG2.2.15, Huh7, NTCP-overexpressing HepG2, and Huh7 cells were cultured in a Dulbecco's modified Eagle's medium (DMEM) containing 10% fetal bovine serum (FBS) and 1% antibiotics at 37° C.

### Experimental Example 2. Preparation of Plasmid

HBV1.2×adr subtype ORF (open reading frame) was prepared in pUC19, and Myc-labeled CP149 ORF (open reading frame) was prepared in pCDNA3. In addition, HBV1.2×adr subtype ORF (open reading frame) was prepared in pAAV for hydrodynamic injection to mouse.

### Experimental Example 3. Quantification of Released Virus

After treating cells with drugs such as ciclopirox, entecavir, and/or tenofovir, the supernatant of the cells was collected. After adding to 30 μL of the supernatant 1×PBS of the same volume, 6 μL of 1 N NaOH was added. After incubation at 37° C. for 1 hour, 6 μL of Tris-HCl/HCl was added. Then, the protein was denatured by heat-treating at 98° C. for 5 minutes. After removing the denatured protein through centrifugation, the virus existing in the supernatant was quantified by real-time PCR.

### Experimental Example 4. Quantification of Virus Remaining Inside

After treating cells with drugs such as ciclopirox, entecavir, and/or tenofovir, the cells were washed with 1×PBS. Then, after lysing the cells and treating with nuclease, HBV DNA was extracted from the cells. The DNA was extracted according to the instruction of the manufacturer (InvivoGen).

### Experimental Example 5. Analysis of HBsAg Protein

The secreted HBsAg protein of HBV was analyzed by enzyme-linked immunosorbent assay. Specifically, HBsAg-specific ELISA was used. A supernatant collected from the cells treated with each drug was analyzed using a kit (hepatitis B surface antigen Ab ELISA kit) according to the instruction of the manufacturer (Abnova).

### Experimental Example 6. Detection of HBV Capsid Protein

An HBV capsid protein was detected by agarose gel electrophoresis. Specifically, the isolated Cp149 dimeric protein was incubated with a core assembly reaction buffer (150 mM NaCl, 15 mM HEPES) and the drug at 37° C. for 1 hour. Then, after separating the protein on agarose gel, immunoblot was conducted using a rabbit polyclonal anti-HBV core antibody. In addition, for isolation of the core expressed in the cells, the cells were lysed with 1% NP-40 and ultracentrifuged (55,000 rpm) at 20° C. for 8 hours. The assembled core that settled down at the bottom was separated on agarose gel and then immunoblot was conducted using a rabbit polyclonal anti-HBV core antibody.

### Example 1. Investigation of HBV-Inhibiting Activity of Ciclopirox

For screening of a drug having inhibitory activity against HBV, an FDA-approved drug library with proven safety was used.

Specifically, after treating HBV-producing HepG2.2.15 cells with about 1,000 drugs at 1 mM every day for 3 days, the quantity of released HBV DNA was measured. After treating the cells with 19 drugs selected through the first screening for 6 days according to the same method, 13 drugs were selected by measuring HBV DNA (second screening). Entecavir, which is used as an HBV drug, was used as a positive control group.

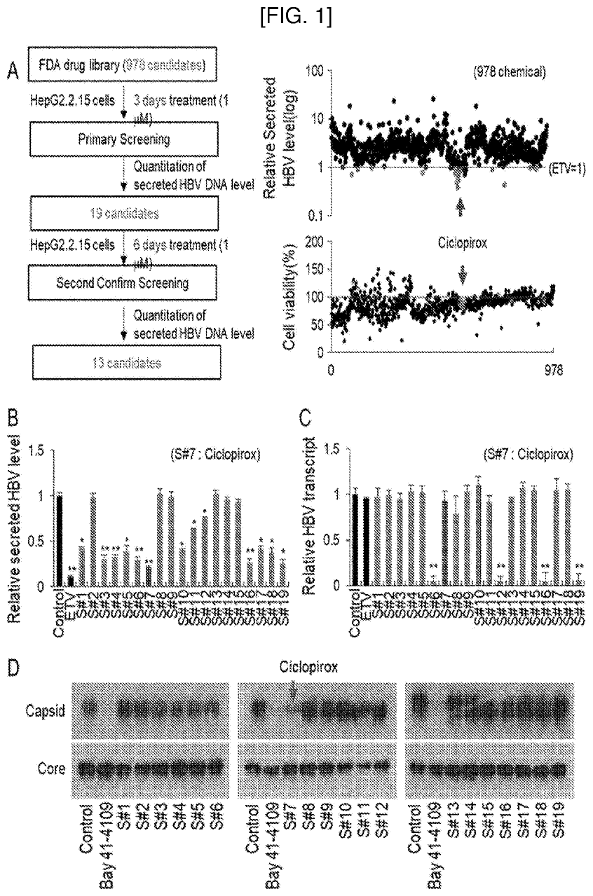

As shown in A and B in FIG. 1, 19 drugs which inhibit the release of HBV DNA more effectively than entecavir were screened, and 13 drugs (#1, #3, #4, #5, #6, #7, #10, #11, #12, #13, #16, #17, #18, #19) among them showed consistent effects for 6 days.

In addition, as can be seen from C in FIG. 1, 4 drugs (#6, #12, #16, #19) among the 19 drugs reduced the expression of HBV transcripts.

Meanwhile, as can be seen from D in FIG. 1, one drug (#7) among the 19 drugs remarkably inhibited the expression of the capsid protein without affecting the expression of the core protein. It was identified to be ciclopirox.

From the results above, it was confirmed that, although ciclopirox does not affect the expression of the core protein since it does not affect the transcription of HBV RNA during the life cycle of HBV, it can be used as a drug exhibiting anti-HBV effect because it reduces the finally released virus DNA by inhibiting the assembly of the capsid protein.

### Example 2. Elucidation of HBV-Inhibiting Mechanism of Ciclopirox

The HBV-inhibiting activity of ciclopirox was identified in Example 1. Since it was confirmed that, whereas ciclopirox does not affect the expression of the core protein but it remarkably inhibits the expression of the capsid protein, it was presumed that ciclopirox inhibits core assembly. The inhibition mechanism was verified in this example.

Specifically, core assembly reaction was analyzed after treating purified core protein 149 (CP149) dimers, core protein-expressing cell lines, entire HBV protein-expressing cell lines, etc. with ciclopirox. As a result, as can be seen from A to C in FIG. 2, it was confirmed that the assembly of the purified core protein 149 (CP149) dimers was inhibited as the concentration of ciclopirox increased, and that core assembly in each cell line was inhibited according to the concentration gradient.

In addition, in order to investigate whether the core assembly is inhibited and the protein remains as dimers, reaction products were separated through ultracentrifugation at different sucrose concentrations after the assembly of CP149 under the assembly reaction conditions. As a result, as can be seen from D in FIG. 2, dimeric CP149 was decreased (fractions 1-3) and core-assembled CP149 was increased (fractions 5-8) when not treated with ciclopirox. In contrast, when treated with ciclopirox, dimeric CP149 was increased and core-assembled CP149 was decreased.

In addition, as can be seen from A and B in FIG. 4, there was no effect on the amount of the released HBsAg protein both in HBV-expressing cell lines and in HBV-overexpressing cell lines.

From the results above, it can be seen that ciclopirox inhibits core assembly only without affecting the expression of the core protein of HBV. Since the inhibition of the core assembly is utilized as a target of virus inhibitors, it can be seen that ciclopirox can be used as a drug exhibiting anti-HBV effect.

### Example 3. Elucidation of HBV Core Protein Binding Site of Ciclopirox

It was confirmed in Example 2 that ciclopirox exhibited an effect of inhibiting the proliferation of HBV in vitro by inhibiting HBV core assembly. Therefore, the ciclopirox binding site of the HBV core protein was investigated. Specifically, structural analysis of the core protein was conducted by forming crystals.

As a result, as can be seen from FIG. 3, the binding site for ciclopirox of the assembled HBV core protein in hexameric form was identified. In particular, it was confirmed through mutation that tyrosine 118 is important for the hydrogen bonding between the drug and the core protein (D in FIG. 3). Through this result, it was demonstrated that ciclopirox exhibits HBV-inhibiting effect by inhibiting core assembly by binding directly to the HBV core protein.

### Example 4. Confirmation of HBV Proliferation-Inhibiting Effect of Ciclopirox

It was confirmed in Examples 1 and 3 that ciclopirox inhibits the core assembly of HBV. Therefore, it was investigated whether it can actually inhibit the proliferation of HBV.

First, as can be seen from A to D in FIG. 5, the quantity of HBV DNA released out of or remaining in cells was decreased in both HBV-expressing cell lines and HBV-overexpressing cell lines as the concentration of ciclopirox was increased.

In addition, after treating HBV-infected liver cancer cell lines NTCP-HepG2 and NTCP-Huh7 with ciclopirox, the degree of HBV proliferation was analyzed. After pretreating the liver cancer cell lines with ciclopirox for 6 hours, the cells were incubated with ciclopirox and HBV for 16 hours. Then, the cells were analyzed after culturing for 14 days. This experimental procedure is schematically illustrated in A in FIG. 6. As shown in B and C in FIG. 6, the NTCP protein expressed from the liver cancer cells NTCP-HepG2 and NTCP-Huh7 was analyzed by immunoblot and flow cytometry.

As a result, as can be seen D to F in FIG. 6, the treatment with ciclopirox resulted in the decrease of HBV DNA released out of the cells and the decrease of HBV cccDNA and rcDNA remaining in the cells according to the concentration gradient (FIG. 6).

From these results, it was found that ciclopirox can effectively inhibit HBV proliferation and can be effective in reducing not only HBV DNA but also cccDNA, which is the biggest problem of the currently used drugs. Accordingly, it was confirmed that ciclopirox can be used as an HBV inhibitor for treating HBV.

### Example 5. Confirmation of Synergistic Effect of Ciclopirox and Entecavir and/or Tenofovir

From Examples 1 to 4, it was confirmed that ciclopirox can inhibit the proliferation of HBV by inhibiting core assembly. Herein, it was investigated whether it exhibits synergistic effect when used in combination with the anti-HBV drug entecavir (ETV) or tenofovir (TDF).

Meanwhile, entecavir (ETV) and tenofovir (TDF) are currently used as drugs that inhibit HBV. However, they have the problem that they cannot remove the cccDNA of HBV. Interferon-based drugs are often used in combination to make up for this problem. Therefore, the synergistic effect of a combination of ciclopirox with the existing drug entecavir or tenofovir was investigated.

Specifically, after treating liver cancer cell lines with ciclopirox alone, with ciclopirox and entecavir, or with ciclopirox tenofovir according to the method of Example 3, the degree of HBV proliferation was investigated. As can be seen from A and B in FIG. 7, the quantity of HBV DNA released out of the cells and HBV DNA remaining in the cells was decreased remarkably when the cells were treated together with ciclopirox and entecavir or tenofovir as compared to when they were treated with ciclopirox alone. Meanwhile, there was no significant decrease in the quantity of the HBsAg protein (C in FIG. 6).

From these results, it was confirmed that, although ciclopirox can effectively inhibit HBV proliferation even alone, it can exhibit better anti-HBV effect when used in combination with entecavir or tenofovir.

### Example 6. Confirmation of HBV Proliferation-Inhibiting Effect of Ciclopirox In Vivo

From Examples 1 to 5, it was confirmed that ciclopirox exhibits superior HBV proliferation-inhibiting effect. Herein, it was investigated whether it exhibits the same effect in vivo.

Specifically, HBV was produced in vivo by hydrodynamically injecting an HBV-expressing plasmid into mouse tail. Then, after treating with ciclopirox alone, tenofovir alone, or a combination of ciclopirox and tenofovir every day for 5 days, the degree of proliferation of HBV in mouse serum was investigated. The experimental procedure is schematically illustrated in A in FIG. 8.

As a result, as can be seen from B in FIG. 8, it was confirmed that the HBV core protein was decreased in the liver cells. In addition, it was confirmed from C in FIG. 8 that ciclopirox remarkably reduces the amount of HBV DNA in the body of mouse when treated alone and the HBV DNA is almost nonexistent 5 days after the treatment. Especially, the HBV DNA-inhibiting effect was slightly superior when ciclopirox was treated together with tenofovir as compared to when ciclopirox was treated alone. Meanwhile, there was no significant difference in the amount of the HBsAg protein (D in FIG. 8).

From these results, it was confirmed that, since ciclopirox can effectively inhibit HBV proliferation in vivo, it can be usefully used as a drug exhibiting anti-HBV effect.

### Example 7. Confirmation of Cytotoxicity of Ciclopirox

From Examples 1 to 6, it was confirmed that ciclopirox exhibits superior HBV proliferation-inhibiting effect in vivo and ex vivo. Herein, the cytotoxicity of ciclopirox was analyzed to investigate whether it can be actually developed into an HBV-inhibiting drug.

As a result, as can be seen from FIG. 9, it was confirmed that decrease in cell viability by ciclopirox was not observed in HBV-expressing cell lines or HBV-overexpressing cell lines. In addition, the cell viability was maintained even at the highest concentration of 10 μM.

From these results, it was confirmed that ciclopirox can be safely used for the human body because it does not affect cell viability, and it can be very effectively used as a drug that exhibits anti-HBV effect.

Based on the above description, it will be understood by those skilled in the art that the present invention may be implemented in a different specific form without changing the technical spirit or essential characteristics thereof. Therefore, it should be understood that the above embodiment is not limitative, but illustrative in all aspects. The scope of the present invention is defined by the appended claims rather than by the description preceding them, and therefore all changes and modifications that fall within metes and bounds of the claims or equivalents of such metes and bounds are therefore intended to be embraced by the claims.

1-10. (canceled)

11. A method for treating a hepatitis B virus (HBV)-induced disease, comprising administering a composition comprising ciclopirox or a pharmaceutically acceptable salt thereof to a subject.

12. The method according to claim 11, wherein the ciclopirox is represented by Chemical Formula 1:

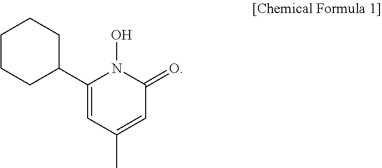

13. The method according to claim 11, wherein the ciclopirox inhibits the assembly of a HBV core protein.

14. The method according to claim 11, wherein the composition further comprises entecavir, tenofovir, or a combination thereof.

15. The method according to claim 11, wherein the HBV-induced disease is hepatitis, hepatocirrhosis, hepatocellular carcinoma, or a combination thereof.

16. A method for inhibiting a hepatitis B virus (HBV), comprising treating a composition comprising ciclopirox or a pharmaceutically acceptable salt thereof to a HBV-expressing cell.

17. The method according to claim 16, wherein the ciclopirox is represented by Chemical Formula 1:

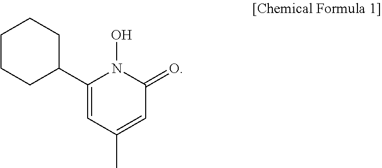

18. The method according to claim 16, wherein the ciclopirox inhibits the assembly of a HBV core protein.

19. The method according to claim 16, wherein the composition further comprises entecavir, tenofovir, or a combination thereof.
